(19) United States
(12) Patent Application Publication
(10) Pub. No.: US 2025/0199001 A1
(43) Pub. Date: Jun. 19, 2025

(54) METHODS OF DETECTION OF INTESTINAL PARASITES

(71) Applicant: IDEXX Laboratories, Inc., Westbrook, ME (US)

(72) Inventors: Melissa Jane Beall, Cape Elizabeth, ME (US); Jesse Buch, Kennebunk, ME (US)

(73) Assignee: IDEXX Laboratories, Inc., Westbrook, ME (US)

(21) Appl. No.: 18/980,022

(22) Filed: Dec. 13, 2024

Related U.S. Application Data
(60) Provisional application No. 63/609,955, filed on Dec. 14, 2023.

(51) Int. Cl. G01N 33/569

(52) U.S. Cl. CPC G01N 33/56905; G01N 2469/10

(57) ABSTRACT

Methods for detecting the presence or absence of an intestinal parasite infection in a mammalian sample are disclosed herein. The methods can be used to confirm the presence or absence of an intestinal parasite by applying a first test that is an immunoassay or a nucleic acid assay and conducting a second test such as microscopic evaluation assay based on the outcome of the first test or based on one or more evaluation metrics. Confirmation of the presence or absence of the intestinal parasite in the mammal can be made, for example, for the purpose of selecting an optimal course of treating the mammal and/or for the purpose of determining whether the mammal has been rid of the infection after treatment has been initiated.

### PRIORITY

This application claims the benefit of U.S. Ser. No. 63/609,955 filed on Dec. 14, 2023, which is incorporated herein by reference in its entirety.

### BACKGROUND

Intestinal parasites are common in animals. If left untreated, they can cause serious disease and even death. Treatment of intestinal parasite infections requires identification of the intestinal parasite. Current methods for diagnosis of intestinal parasite infections primarily involve microscopic examination of fecal samples, either directly in fecal smears or following concentration of ova by flotation in density media. Despite this procedure's high adoption, it can be labor, time, and cost intensive. In addition, the accuracy of results of these methods is highly dependent upon the skill and expertise of the operator. There is therefore a need in the art for identifying and optimizing high throughput diagnostic methods, which can reduce the number of samples requiring microscopic evaluation.

### SUMMARY

Provided herein are methods for detecting an intestinal parasite infection in a mammal. Such methods can include a) obtaining a fecal sample from the mammal; b) providing a panel of tests, comprising a first test and a second test, wherein the first test is an immunoassay or a nucleic acid assay having a first threshold value and the second test is a microscopic evaluation assay; c) conducting the first test and determining a signal output for the first test; d) conducting the second test if the signal output is greater than the first threshold value and/or if the mammal meets at least one evaluation metric; and/or e) determining that the mammal has an intestinal parasite infection if the signal output from the first test is greater than the first threshold value, if the second test yields a positive result, or both.

In an aspect, a method is provided for reducing an amount of fecal samples in a group of fecal samples required to be subjected to intestinal parasite microscopic evaluation assays during the process of determining if a sample indicates an intestinal parasite infection. The method can comprise (a) obtaining a group of fecal samples from mammals and, for each fecal sample in the group of fecal samples (b) providing a panel of tests comprising a first test and a second test, wherein the first test is an immunoassay or a nucleic acid assay having a first threshold value and the second test is a microscopic evaluation assay; (c) conducting the first test and determining a signal output; (d) conducting the second test if the signal output is greater than the first threshold value or if the mammal meets at least one evaluation metric; and (e) determining that the mammal has an intestinal parasite infection if the signal output from the first test is greater than the first threshold value, if the second test yields a positive result, or both the signal output from the first test is greater than the first threshold value and the second test yields a positive result. The amount of fecal samples in the group of fecal samples subjected to intestinal parasite microscopic evaluation assays can be reduced as compared to methods that do not include steps (b)-(e).

In an aspect, a method of monitoring treatment of an intestinal parasite in a mammal. The method can comprise obtaining a fecal sample from the mammal that has been treated for an intestinal parasite. A panel of tests is provided comprising a first test and a second test, wherein the first test is an immunoassay or a nucleic acid assay having a first threshold value and the second test is a microscopic evaluation assay. The first test can be conducted and a signal output from the first test can be determined. The second test is conducted if the signal output from the first test is greater than the first threshold value or if the mammal meets at least one evaluation metric. It is determined that the mammal still has an intestinal parasite infection if the signal output from the first test is greater than the first threshold value, if the second test yields a positive result, or both the signal output from the first test is greater than the first threshold value and the second test yields a positive result. Where it is determined that the mammal still has an intestinal parasite infection, one or more treatments for intestinal parasite infection can be provided to the mammal.

In some aspects, the first threshold value can be a low threshold value. The first test can further have a second threshold value. In some aspects, the second threshold value can be a high threshold value. In some aspects, the ratio of the second threshold value and the first threshold value is at least 1.

The evaluation metric can include the age of the mammal, species of the mammal, breed of the mammal, incidence of an intestinal parasite in a population of the mammal, the time of year when the test is performed on the mammal, the level of care experienced by the mammal, one or more symptoms that are consistent with intestinal parasite infection, and/or the geographical location of the mammal.

In some aspects, the methods can be used to determine that the mammal does not have an intestinal parasite infection if the signal output from the first test is less than the first threshold value. In some aspects, the methods can be used to determine that the mammal has an intestinal parasite infection if the signal output from the first test is greater than the first threshold value and the second test yields a positive result. In some aspects, the methods can be used to determine that the mammal has an intestinal parasite infection if the signal output from the first test is greater than the first threshold and the second test yields a negative result. In some aspects, the methods can be used to determine that the mammal has an intestinal parasite infection if the signal output from the first test is less than the first threshold value, but the mammal meets at least one evaluation metric and the second test yields a positive result. In some aspects, the methods can be used to determine that the mammal does not have an intestinal parasite infection if the signal output from the first test is less than the first threshold value but the mammal meets at least one evaluation metric and the second test yields a negative test result. In some aspects, the methods can be used to determine that the mammal does not have an intestinal parasite infection if the signal output from the first test is greater than the first threshold value, but less than the second threshold value and the second test yields a negative result. In some aspects, the methods can be used to determine that the mammal has an intestinal parasite infection if the signal output from the first test is greater than both the first threshold value and the second threshold value and the second test yields a positive result. In some aspects, the methods can be used to determine that the mammal has an intestinal parasite infection if the signal output from the first test is greater than both the first threshold value and the second threshold value and the second test yields a negative result. In some aspects, the methods can be used to determine that the mammal has an intestinal parasite infection if the signal output from the first test is greater than the first threshold value but less than the second threshold value and the second test yields a positive result.

In some aspects, the intestinal parasite infection can be an infection with an intestinal parasite such as, but not limited to, one or more nematodes, one or more cestodes, one or more protozoa or a combination thereof. In some aspects, the nematode is a hookworm, a roundworm, or a whipworm, the cestode is a tapeworm and the protozoan is a Giardia, Cystoisospora, or Cryptosporidium.

In some aspects, the first test can include a sub panel of tests.

In some aspects, the first test can be a multiplex assay.

In some aspects, the first threshold value and the second threshold value can be calculated for each intestinal parasite in the first test.

In some aspects, the roundworm can be Toxocara canis, Toxocara cati, Toxocara vitulorum, Toxascaris leonina, Baylisascaris procyonis, Ascaridia galli, Parascaris equorum, Ascaris suum, Ascaris lumbricoides, Anisakis simplex, and/or Pseudoterranova decipiens. In some aspects, the whipworm can be Trichuris vulpis, Trichuris campanula, Trichuris serrata, Trichuris suis, Trichuris trichiura, Trichuris discolor and/or Trichocephalus trichiuris. In some aspects, the hookworm can be Ancylostoma caninum, Ancylostoma braziliense, Ancylostoma duodenale, Ancylostoma ceylanicum, Ancylostoma tubaeforme, Ancylostoma pluridentatum, Necator americanus, or Uncinaria stenocephala. In some aspects, the Giardia can be Giardia duodenalis, which is alternatively referred to as Giardia lamblia. In some aspects, the tapeworm can be Taenia pisiformis, Taenia taeniaeformis, Taenia crassiceps or Dipylidium caninum. In some aspects, the Cystoisospora can be Cystoisospora canis, Cystoisospora ohioensis, Cystoisospora burrowsi, Cystoisospora neorivolta, Cystoisospora felis, or Cystoisospora rivolta. In some aspects, the Cryptosporidium can be Cryptosporidium parvum, Cryptosporidium canis, or Cryptosporidium felis.

In some aspects, the first test can be an immunoassay that includes one or more steps such as a) contacting the fecal sample with one or more antibodies that can specifically bind at least one antigen of the intestinal parasite; b) an antibody-antigen complex forming in the presence of the antigen of the intestinal parasite, if any, in the fecal sample; and/or c) detecting the presence or absence of antibody-antigen complex by determining the signal output. In some aspects, detecting the presence or absence of antibody-antigen complex can include a step of providing at least one secondary antibody that binds to the complex. In some aspects, the one or more antibodies or the secondary antibody can be coupled to a fluorescent label. In some aspects, the one or more antibodies or the secondary antibody can be attached to a substrate or a solid support. In some aspects, the substrate or solid support in the immunoassay can include an epoxy-based resin surface. In some aspects, the substrate or solid support can be a film, a microbead; a microparticle; a micro pellet; a micro wafer; a paramagnetic bead; a microparticle containing a bar code; a paramagnetic microparticle; a microparticle containing a bar code; a paramagnetic microparticle containing a bar code; or a bead containing a nickel bar code.

In some aspects, the nucleic acid assay can include determining presence or absence of a particular nucleic acid molecule from the intestinal parasite.

In some aspects, the nucleic acid assay can be a polymerase chain reaction (PCR)-based assay. In some aspects, the one or more nucleic acid primers and/or nucleic acid probes attached to a substrate or solid support.

In some aspects, the substrate or solid support can include an epoxy-based resin surface. In some aspects, the substrate or solid support can be a film, a microbead; a microparticle; a micro pellet; a micro wafer; a paramagnetic bead; a microparticle containing a bar code; a paramagnetic microparticle; a microparticle containing a bar code; a paramagnetic microparticle containing a bar code; or a bead containing a nickel bar code. In some aspects, the substrate, the nucleic acid probe and/or the nucleic acid primer can be coupled to a fluorescent label.

In some aspects, the second test further can further include concentrating the intestinal parasite or eggs, prior to the microscopic evaluation assay. In some aspects, the second test is capable of detecting a parasite species not detectable by the first test. In some aspects, the second test can include determining the presence or absence of an anthelmintic resistance marker. In some aspects, the anthelmintic resistance marker is a mutation. In some aspects, the anthelmintic resistance marker is a haplotype.

In some aspects, the methods of the disclosure can further include administering a therapeutic agent to treat the mammal having an intestinal parasite infection. A treatment can comprise administration of albendazole, ivermectin, febantel, fenbendazole, milbemycin, milbemycin oxime, moxidectin, piperazine, pyrantel, emodepside, eprinomectin, praziquantel, selamectin, oxibendazole, mebendazole, menbendazole, nitroscanate, afoxolaner, pamoate, praziquantel, imidacloprid, oxantel, afoxolaner, lufenuron, praziquantel, epsiprantel, fipronil, methoprene, metronidazole, chloroquine, paromomycin, tinidazole, secnidazole, or a combination thereof.

### DETAILED DESCRIPTION

The details of one or more variations of the subject matter described herein are set forth in the description below. Other features and advantages of the subject matter described herein will be apparent from the description and from the claims. The disclosed subject matter is not, however, limited to any particular aspect disclosed.

### Overview

Identification of intestinal parasites and the diagnosis of an intestinal parasitic infection is needed to determine the therapeutic strategy for treating a particular infection. Current methods for diagnosis of intestinal parasites primarily involve microscopic examination of fecal samples.

Stool handling for microscopic examination can be disagreeable and hazardous. Sanitary and inoffensive procedures for processing stool are awkward and often complex. Such procedures can include weighing, centrifuging, and storing of fecal samples and are difficult except in a clinical laboratory equipped with a suitable apparatus, protective equipment, and a skilled technician. Therefore, any reduction in the number of samples requiring a microscopic evaluation and/or any reduction in contact between test operator and the test material can be desirable.

Approximately 80% of the fecal samples submitted to a U.S. commercial reference laboratory and evaluated by microscopic assay also tend to have no organisms of clinical importance, such as intestinal parasites. Because centrifugal flotation, a pre-processing step in the microscopic evaluation assay, is costly from a time, labor, materials, and waste perspective, there is a need in the art to combine microscopic evaluation assays with one or more high throughput diagnostic methods. This strategy could reduce the number of samples requiring microscopic evaluation.

The present disclosure provides improved methods of detection of intestinal parasites. Standard methods in the art perform microscopic evaluation of intestinal parasite alone or in parallel with immunoassays or nucleic acid assays. The methods described herein include a first test comprising, for example, one or more immunoassays or nucleic acid assays. Based on the outcome of the first test, a second test such as a microscopic evaluation assay is performed. By tuning the sensitivity of the first test, the present disclosure reduces the number of samples requiring the second test, i.e., the microscopic evaluation.

### Samples

In some aspects, a sample can be a fecal sample. The sample can be a fecal sample from a mammal. The term “fecal sample”, as used herein, includes feces, any sample containing feces, fractions of feces, concentrates of feces, and extracts of feces.

The sample can be taken directly from the mammal, or the sample can be taken from anything that has been in contact with the mammal, including environmental samples. A fecal sample can be collected by, for example, perianal swab. The fecal sample can be obtained from the large intestine, cecum, colon, rectum, the anus or any portion of the gastrointestinal tract. The fecal sample can be fresh or decaying fecal droppings from the mammal. As another example, the fecal sample can include soil, dirt, sand, plant material, pollen, or any other material that can be mixed with feces.

A soluble portion of the fecal sample can be used in the methods described herein. The soluble portions of the sample can be collected by using filtration, extraction, centrifugation, or simple mixing followed by gravimetric settling.

In some aspects, the sample can be a non-fecal sample. For example, the sample can be obtained by swabbing the mammal, such as the oral cavity of the mammal. As another example, tissue sections, including tissue from small intestine, large intestine, cecum, colon, rectum, or another tissue of the gastrointestinal tract, can be obtained by biopsy. Samples can be obtained from any mammal, including, e.g., dogs, cats, human, non-humans, non-human primates, bovines, equines, etc.

### Intestinal Parasite Infection

The methods described herein can be used to detect an intestinal parasite infection. An intestinal parasite infection can be caused by one or more types of intestinal parasites (e.g., 1, 2, 3, 4 or more types of intestinal parasites).

The methods can comprise a first test that is designed to detect to an intestinal parasite. An intestinal parasite can be one or more nematodes, one or more cestodes, one or more protozoans or a combination thereof. A nematode can be, for example, a hookworm, a roundworm, or a whipworm, a cestode can be, for example, a tapeworm, and a protozoan can be, for example, a Giardia, a Cystoisospora, ora Cryptosporidium.

A round worm can be, for example, Toxocara canis, Toxocara cati, Toxocara vitulorum, Toxascaris leonina, Baylisascaris procyonis, Ascaridia galli, Parascaris equorum, Ascaris suum, Ascaris lumbricoides, Anisakis simplex, and/or Pseudoterranova decipiens.

A whipworm can be, for example, Trichuris vulpis, Trichuris campanula, Trichuris serrata, Trichuris suis, Trichuris trichiura, Trichuris discolor and/or Trichocephalus trichiuris.

A hookworm can be for example, Ancylostoma caninum, Ancylostoma braziliense, Ancylostoma duodenal, Ancylostoma ceylanicum, Ancylostoma tubaeforme, Ancylostoma pluridentatum, Necator americanus, and/or Uncinaria stenocephala.

An intestinal parasite can be, for example, Giardia lamblia. A Cystoisospora can be, for example, Cystoisospora canis, Cystoisospora ohioensis, Cystoisospora burrowsi, Cystoisospora neorivolta, Cystoisospora felis, or Cystoisospora rivolta. A Cryptosporidium can be, for example, Cryptosporidium parvum, Cryptosporidium canis, or Cryptosporidium felis.

A tapeworm can be for example, Taenia pisiformis, Taenia taeniaeformis, Taenia crassiceps and/or Dipylidium caninum.

The methods can include a second test, which is a microscopic evaluation assay. A microscopic evaluation assay can be used to detect any intestinal parasite. An intestinal parasite detected by the second test can be, for example, a hookworm, a Giardia, a Cystoisospora, a Cryptosporidium, a roundworm, a whipworm, and/or a tapeworm. A microscopic evaluation assay can detect an intestinal parasite that is not detectable by the first test, e.g., an intestinal parasite other than a hookworm, a Giardia, a Cystoisospora, a Cryptosporidium, a roundworm, a whipworm, and/or a tapeworm.

### Workflow

The present disclosure provides methods to detect an intestinal parasite infection. In some aspects, methods for reducing an amount of fecal samples in a group of fecal samples required to be subjected to intestinal parasite microscopic evaluation assays is provided. Determining the presence of intestinal parasites can require evaluation of fecal samples by microscopic assay. Microscopic assays are costly in terms of time, labor, and materials. If the number of fecal samples that need to be completed to determine intestinal parasite infection could be reduced, then a great cost savings could be realized for the laboratory, which could be passed onto the consumer. The methods described herein can reduce the number of fecal samples needing to be examined via microscopic evaluation assays can be reduced by 5, 10, 15, 20, 30, 40, 50, 60, 70% or more. A group of fecal samples can comprise 5, 10, 50, 100, 200, 500, 750, 1,000 or more samples from a group of the same species of patient or from a group of different species of patients.

The methods can include testing the fecal sample using a panel of tests. The panel of tests can be incorporated into a workflow that includes a first test and a second test. The first test can be an immunoassay or a nucleic acid assay. The first test can further include a sub panel of tests with each sub panel designed to test for the presence of at least one type of intestinal parasite. In an aspect, the first test can be a multiplex assay that can simultaneously test for the presence of more than one intestinal parasite (e.g., 2, 3, 4, 5, 6, 7, 8, or more different types of intestinal parasites. Therefore, a sub panel is a test that is specific for one type of parasite.

In some aspects, the yield of the first test, e.g., the formation of the antigen-antibody complex in the immunoassay or the nucleic acid probe/primer-target nucleic acid molecule complex in the nucleic acid assay can be detected using a detection agent. The detection agent can be coupled to an antibody in the immunoassay and/or the nucleic acid probe/primer in the nucleic acid assay or the complex formed in the assays. In some aspects, in the first test the detection agent can be coupled to a substrate or solid support to which the antibody or the nucleic acid probe is attached to. The yield of the first test can be estimated as the quantity of the detection agent measured in the assay and is herein referred to as the “signal output.” A detection agent can be, for example, a fluorescent label, and the signal output can be quantified as median value of the fluorescence intensity (also referred to herein as the “median fluorescence intensity” (MFI)). The signal output can be normalized by subtracting the signal output from the MFI of a negative control sample.

An MFI can indicate the quantity of the target analyte (i.e., an intestinal parasite protein or antigen) bound by an antibody in an immunoassay or the quantity of a target analyte nucleic acid molecule (e.g., a nucleic acid molecule derived from an intestinal parasite). A higher MFI value corresponds to a higher concentration of the target analyte present in the sample. MFI can be reported in Relative Fluorescence Units (RFU), which indicates the relative amount of fluorescence detected as compared to a standard curve. Known concentrations of antigen or nucleic acid molecules are used to produce a standard curve and then this data is used to measure the concentration of unknown samples by comparison to the linear portion of the standard curve.

The signal output from the first test can have a threshold value. As used herein, the term “threshold value” refers to the value of the signal output, beyond which, the signal output changes from being considered a negative result for the first test to being considered a positive result for the first test. In some aspects, a signal output greater than a threshold value is not reflective of the presence of intestinal parasite infection in a sample. One or more threshold values can be instituted in the first test. In an aspect, the threshold values can be estimated based on results of experimental infections prior to evidence of patency (i.e., detectable parasite infection). The first test can have a first threshold value and a second threshold value. The first threshold value can be a low threshold value that is set to increase the sensitivity of the first test. A ratio of the second threshold value and the first threshold value can be at least 1, at least 2, at least 3, at least 4, at least 5 or more. The first threshold value can result in a sensitivity of the first test of from about 85% to about 100% (e.g., about 85, 86, 87, 88, 89, 90, 91, 92, 93, 94, 95, 96, 97, 98, 99, or 100% (or any range between about 50% and 100%, or between about 70% and 100%, or between about 85 and 100%). Sensitivity (i.e., true positive rate) is the probability of a positive test result, conditioned on the individual truly being positive. The second threshold value can be a high threshold value that is set to increase the specificity of the first test. The second threshold value can result in a specificity of the first test of about 85% to about 100% (e.g., about 85, 86, 87, 88, 89, 90, 91, 92, 93, 94, 95, 96, 97, 98, 99, or 100% (or any range between about 50% and 100%, or between about 70% and 100%, or between about 85 and 100%). Specificity (i.e., true negative rate) is the probability of a negative test result, conditioned on the individual truly being negative.

A first threshold value can be based on a signal output and defined as a percentage of the assay dynamic range of about 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. A dynamic range is the ratio of the highest measurable output of an assay to the lowest measurable output. That is, the range of concentrations or signal intensities that an assay can measure accurately.

In some aspects a first threshold value can have a signal output such as an MFI of about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000 RFU or more.

In some aspects, a first threshold value can be based on a signal output and defined as a range of percentage of the assay dynamic range, such as, but not limited to, 0.000001%-0.00001%, 0.00001%-0.0001%, 0.0001%-0.001%, 0.001%-0.01%, 0.01%-0.1%, 0.1%-1%, 1%-2%, 2%-3%, 3%-4%, 4%-5%, 5%-6%, 6%-7%, 7%-8%, 8%-9%, 9%-10%, 10%-11%, 11%-12%, 12%-13%, 13%-14%, 14%-15%, 15%-16%, 16%-17%, 17%-18%, 18%-19%, 19%-20%, 20%-21%, 21%-22%, 22%-23%, 23%-24%, 24%-25%, 25%-26%, 26%-27%, 27%-28%, 28%-29%, 29%-30%, 30%-31%, 31%-32%, 32%-33%, 33%-34%, 34%-35%, 35%-36%, 36%-37%, 37%-38%, 38%-39%, 39%-40%, 40%-41%, 41%-42%, 42%-43%, 43%-44%, 44%-45%, 45%-46%, 46%-47%, 47%-48%, 48%-49%, 49%-50%, 50%-51%, 51%-52%, 52%-53%, 53%-54%, 54%-55%, 55%-56%, 56%-57%, 57%-58%, 58%-59%, 59%-60%, 60%-61%, 61%-62%, 62%-63%, 63%-64%, 64%-65%, 65%-66%, 66%-67%, 67%-68%, 68%-69%, 69%-70%, 70%-71%, 71%-72%, 72%-73%, 73%-74%, 74%-75%, 75%-76%, 76%-77%, 77%-78%, 78%-79%, 79%-80%, 80%-81%, 81%-82%, 82%-83%, 83%-84%, 84%-85%, 85%-86%, 86%-87%, 87%-88%, 88%-89%, 89%-90%, 90%-91%, 91%-92%, 92%-93%, 93%-94%, 94%-95%, 95%-96%, 96%-97%, 97%-98%, 98%-99%

In some aspects, a first threshold value can be based on a signal output such as an MFI selected from a range, such as, but not limited to, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000, 5000-5100, 5100-5200, 5200-5300, 5300-5400, 5400-5500, 5500-5600, 5600-5700, 5700-5800, 5800-5900, 5900-6000, 6000-6100, 6100-6200, 6200-6300, 6300-6400, 6400-6500, 6500-6600, 6600-6700, 6700-6800, 6800-6900, 6900-7000, 7000-7100, 7100-7200, 7200-7300, 7300-7400, 7400-7500, 7500-7600, 7600-7700, 7700-7800, 7800-7900, 7900-8000, 8000-8100, 8100-8200, 8200-8300, 8300-8400, 8400-8500, 8500-8600, 8600-8700, 8700-8800, 8800-8900, 8900-9000, 9000-9100, 9100-9200, 9200-9300, 9300-9400, 9400-9500, 9500-9600, 9600-9700, 9700-9800, 9800-9900, 9900-10000, 10000-11000, 11000-12000, 12000-13000, 13000-14000, 14000-15000, 15000-16000, 16000-17000, 17000-18000, 18000-19000, 19000-20000, 20000-21000, 21000-22000, 22000-23000, 23000-24000, 24000-25000, 25000-26000, 26000-27000, 27000-28000, 28000-29000, 29000-30000, 30000-31000, 31000-32000, 32000-33000, 33000-34000, 34000-35000, 35000-36000, 36000-37000, 37000-38000, 38000-39000, 39000-40000, 40000-41000, 41000-42000, 42000-43000, 43000-44000, 44000-45000, 45000-46000 RFU or higher.

In some aspects a second threshold value can be based on a signal output and defined as a percentage of the assay dynamic range of about 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9%

In some aspects a second threshold value can have a signal output such as an MFI of about 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000 RFU or more.

In some aspects a second threshold value can be based on a signal output and defined as a range of percentage of the assay dynamic range of about, such as, but not limited to, 0.00001%-0.0001%, 0.0001%-0.001%, 0.001%-0.01%, 0.01%-0.1%, 0.1%-1%, 1%-2%, 2%-3%, 3%-4%, 4%-5%, 5%-6%, 6%-7%, 7%-8%, 8%-9%, 9%-10%, 10%-11%, 11%-12%, 12%-13%, 13%-14%, 14%-15%, 15%-16%, 16%-17%, 17%-18%, 18%-19%, 19%-20%, 20%-21%, 21%-22%, 22%-23%, 23%-24%, 24%-25%, 25%-26%, 26%-27%, 27%-28%, 28%-29%, 29%-30%, 30%-31%, 31%-32%, 32%-33%, 33%-34%, 34%-35%, 35%-36%, 36%-37%, 37%-38%, 38%-39%, 39%-40%, 40%-41%, 41%-42%, 42%-43%, 43%-44%, 44%-45%, 45%-46%, 46%-47%, 47%-48%, 48%-49%, 49%-50%, 50%-51%, 51%-52%, 52%-53%, 53%-54%, 54%-55%, 55%-56%, 56%-57%, 57%-58%, 58%-59%, 59%-60%, 60%-61%, 61%-62%, 62%-63%, 63%-64%, 64%-65%, 65%-66%, 66%-67%, 67%-68%, 68%-69%, 69%-70%, 70%-71%, 71%-72%, 72%-73%, 73%-74%, 74%-75%, 75%-76%, 76%-77%, 77%-78%, 78%-79%, 79%-80%, 80%-81%, 81%-82%, 82%-83%, 83%-84%, 84%-85%, 85%-86%, 86%-87%, 87%-88%, 88%-89%, 89%-90%, 90%-91%, 91%-92%, 92%-93%, 93%-94%, 94%-95%, 95%-96%, 96%-97%, 97%-98%, 98%-99%, 99%-99.9%.

In some aspects a second threshold value can be a signal output, such as an MFI selected from a range, such as, but not limited to, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000, 5000-5100, 5100-5200, 5200-5300, 5300-5400, 5400-5500, 5500-5600, 5600-5700, 5700-5800, 5800-5900, 5900-6000, 6000-6100, 6100-6200, 6200-6300, 6300-6400, 6400-6500, 6500-6600, 6600-6700, 6700-6800, 6800-6900, 6900-7000, 7000-7100, 7100-7200, 7200-7300, 7300-7400, 7400-7500, 7500-7600, 7600-7700, 7700-7800, 7800-7900, 7900-8000, 8000-8100, 8100-8200, 8200-8300, 8300-8400, 8400-8500, 8500-8600, 8600-8700, 8700-8800, 8800-8900, 8900-9000, 9000-9100, 9100-9200, 9200-9300, 9300-9400, 9400-9500, 9500-9600, 9600-9700, 9700-9800, 9800-9900, 9900-10000, 10000-11000, 11000-12000, 12000-13000, 13000-14000, 14000-15000, 15000-16000, 16000-17000, 17000-18000, 18000-19000, 19000-20000, 20000-21000, 21000-22000, 22000-23000, 23000-24000, 24000-25000, 25000-26000, 26000-27000, 27000-28000, 28000-29000, 29000-30000, 30000-31000, 31000-32000, 32000-33000, 33000-34000, 34000-35000, 35000-36000, 36000-37000, 37000-38000, 38000-39000, 39000-40000, 40000-41000, 41000-42000, 42000-43000, 43000-44000, 44000-45000, 45000-46000 RFU or higher.

A signal output can be measured at various time points throughout an assay. Thee time points can be PCR amplification cycles. The signal output can be below the threshold value at a particular amplification cycle, but it can be above the threshold value at a later amplification cycle.

A first threshold of a PCR assay can be based on a signal output and defined as the fluorescence intensity. The first threshold of a PCR assay can be based on a signal output and defined as the fluorescence intensity at a particular amplification cycle, e.g., at cycle 25, 28, 30, 33, 35, 38 or 41.

A second threshold of a PCR assay can be based on a signal output and defined as the fluorescence intensity. The second threshold of a PCR assay can be based on a signal output and defined as the fluorescence intensity at a particular amplification cycle, e.g., at cycle 25, 28, 30, 33, 35, 38 or 41.

The first threshold value or the second threshold value can be defined as the number of amplification cycles, such as, but not limited to, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 21 cycles, 22 cycles, 23 cycles, 24 cycles, 25 cycles, 26 cycles, 27 cycles, 28 cycles, 29 cycles, 30 cycles, 31 cycles, 32 cycles, 33 cycles, 34 cycles, 35 cycles, 36 cycles, 37 cycles, 38 cycles, 39 cycles, 40 cycles, 41 cycles, 42 cycles, 43 cycles, 44 cycles, 45 cycles, 46 cycles, 47 cycles, 48 cycles, 49 cycles, 50 cycles, 51 cycles, 52 cycles, 53 cycles, 54 cycles, 55 cycles, 56 cycles, 57 cycles, 58 cycles, 59 cycles, 60 cycles, 61 cycles, 62 cycles, 63 cycles, 64 cycles, 65 cycles, 66 cycles, 67 cycles, 68 cycles, 69 cycles, 70 cycles, or more.

The sensitivity of a PCR assay can increase with the number of amplification cycles, i.e., observing the signal output at a higher reaction cycle number can result in a higher sensitivity of the assay. For example, the amplification product may not be detectable above background at cycle 25, but it may be detectable above background at cycle 35.

In an aspect, a threshold value can be defined as the lowest number of amplification cycles at which a signal output that is considered positive is measured.

In an aspect, the sensitivity of a PCR assay can be inversely correlated with the number of amplification cycles, i.e., observing the signal output at a higher reaction cycle number can result in a higher sensitivity of the assay. For example, the amplification product may not be detectable above background at cycle 25, but it may be detectable above background at cycle 35.

The first threshold in a PCR reaction can be a high cycle number (e.g., 30, 35, 40 or more cycles) that is set to increase the sensitivity of the first test. The second threshold in a PCR reaction can be a low cycle number (e.g., 28, 25, 20 or fewer cycles) that is set to increase the specificity of the first test.

The first threshold in a PCR reaction can be a low primer and/or probe annealing temperature that is set to increase the sensitivity of the first test. In general, this means using a lower annealing temperature, e.g., one that is below the melting temperature (Tm) of the lowest primer pair or probe. The second threshold in a PCR reaction can be a high primer and/or probe annealing temperature that is set higher than that of the first threshold to increase the specificity of the first test. In general, this means raising the annealing temperature as close as possible to the Tm of the primers and probes. That is, 1, 2, 3, 4 or more degrees below the lowest Tm of the primer pair or probe.

Several approaches are available for optimizing or modifying the sensitivity or specificity of PCR reactions can be used. Such approaches include strategies for balancing the sensitivity of individual assays that are part of a multiplexed assay. Approaches and factors for the optimization of PCR reactions and modulation of their sensitivity have been described in the literature, e.g., Sint D, et al., Advances in multiplex PCR: balancing primer efficiencies and improving detection success. Methods Ecol Evol. 2012 October; 3(5):898-905. doi: 10.1111/j.2041-210X.2012.00215.x. PMID: 23549328; PMCID: PMC3573865, and Markoulatos P et al., Multiplex polymerase chain reaction: a practical approach. J Clin Lab Anal. 2002 16(1):47-51. doi: 10.1002/jcla.2058. PMID: 11835531; PMCID: PMC6808141, (the contents of each of which are herein incorporated by reference in its entirety).

The sensitivity of a PCR assay can be increased by optimizing the design and/or concentration of one or more primers or probes. The sensitivity of a PCR assay can be decreased by modifying the design and/or concentration of one or more primers or probes.

In aspect, the sensitivity of the assay can be increased by optimizing the concentration of dNTPs and/or magnesium ions (Mg²⁺) in the PCR assay solution. In an aspect, the sensitivity of the assay can be decreased by modifying the concentration of dNTPs and/or magnesium ions (Mg²⁺) in the PCR assay solution.

In an aspect, the sensitivity of a PCR assay can be increased by raising the amount of input nucleic acid molecules from the sample, i.e., by increasing the amount of template nucleic acid molecules in the reaction mix. In an aspect, the sensitivity of a PCR assay can be decreased by lowering the amount of input nucleic acid molecules from the sample, i.e., by decreasing the amount of template nucleic acid molecules in the reaction mix.

According to the present disclosure the workflow for detecting intestinal parasite infections can involve obtaining a fecal sample from a mammal and providing a panel of tests that include a first test and a second test. The first test can be conducted and a signal output from the first test can be determined. In an aspect, the second test can be conducted if the signal output of the first test is greater than the first threshold value and/or if the mammal meets at least one evaluation metric. The first threshold value can be a low threshold value or a sensitive threshold value. Threshold values for an immunoassay or a nucleic acid assay can be set to high threshold values to maximize specificity of the immunoassay or nucleic acid assay and potentially avoid false positive results. The first threshold value can also be set to a low value to enhance sensitivity of the first test to capture as many samples as possible with potential intestinal parasite infection that can be further evaluated using the second test. In an aspect, the first test can include a second threshold value which is the high threshold value.

In an aspect, an evaluation metric for the mammal can include the age of the mammal, species of the mammal, breed of the mammal, incidence of an intestinal parasite in a population of the mammal, the time of year when the test is performed on the mammal, the level of care experienced by the mammal, one or more symptoms that are consistent with intestinal parasite infection, and/or the geographical location of the mammal. The significance of several evaluation metrics in cats and dogs is described in Sweet et al., Retrospective analysis of feline intestinal parasites: trends in testing positivity by age, USA geographical region and reason for veterinary visit. Parasit Vectors. 2020 Sep. 15; 13(1):473. doi: 10.1186/s13071-020-04319-4, and in Sweet et al., A 3-year retrospective analysis of canine intestinal parasites: fecal testing positivity by age, U.S. geographical region and reason for veterinary visit. Parasit Vectors. 2021 Mar. 20; 14(1):173. doi: 10.1186/s13071-021-04678-6 (the contents of each of which are herein incorporated by reference in its entirety). If a mammal to be tested meets one or more evaluation metrics, then the mammal is considered to meet at least one evaluation metric.

In an aspect, the evaluation metric is the age of the mammal. A mammal can be at an increased risk of having intestinal parasites if the mammal is less than 12 months old; a mammal can be at an increased risk of having intestinal parasites if the mammal is less than 2 years old; and a mammal can be at an increased risk of having intestinal parasites if the mammal is over 10 years of age. A dog has a high likelihood of roundworm and hookworm infection at less than 8 weeks of age. Cats and dogs less than 12 months of age have a higher chance of Cystoisospora and Giardia infection than dogs and cats of 12 months or older. Therefore, a young or old mammal would meet at least one evaluation metric.

In an aspect, the evaluation metric is the species of the mammal. For example, cats are more likely than dogs to test positive for tapeworm. Therefore, a cat would meet at least one evaluation metric if tapeworm is being assayed.

In an aspect, the evaluation metric is the breed of the mammal. For instance, the greyhound breed of dogs is more likely to be infected with hookworm than dogs of other breeds. Therefore, a greyhound would meet at least one evaluation metric if hookworm is being assayed.

In an aspect, the evaluation metric is the incidence of an intestinal parasite in a population of the mammal. A mammal that is a member of a population in which one or more other mammals are known to have an intestinal parasite infection, is at an increased risk of having an intestinal parasite infection. Such populations can include members of a kennel, a shelter, a building, a play group, a dog park, a breeding operation, a puppy mill, or any other grouping of mammals that allows its members to interact directly or through bodily excretions. Therefore, a mammal would meet at least one evaluation metric where the mammal is a member of a population in which one or more other mammals are known to have an intestinal parasite infection or is present in groups with other mammals.

In an aspect, the evaluation metric is the time of year when the test is performed on the mammal. For instance, infections with hookworm, roundworm, and/or whipworm are more prevalent from December to February and from July to August. Infections with tapeworm are more prevalent when fleas are most active, i.e., typically in summer and fall. The seasonality of certain intestinal parasites is described in Drake & Carey, Seasonality and changing prevalence of common canine gastrointestinal nematodes in the USA. Parasites Vectors 12, 430 (2019) doi.org/10.1186/s13071-019-3701-7 (the contents of which are herein incorporated by reference in its entirety). Therefore, a mammal would meet at least one evaluation metric if hookworm, roundworm, and/or whipworm are being assayed and the sample was collected in December to February or July to August. Additionally, a mammal would meet at least one evaluation metric if tapeworm is being assayed and the sample was collected in summer or fall.

In an aspect, the evaluation metric is the geographical location of the mammal. The geographical location can be an area where the mammal primarily resides, an area the mammal has visited, or both. Tapeworm infections are more common in geographical areas where fleas are endemic. Cats or dogs that have had access to the outdoors and have been allowed to hunt are more likely to be infected with tapeworm. Cats, in general, are more likely than dogs to test positive for tapeworm. As for the hookworm, Uncinaria is primarily found in northern regions of both US and Europe, while Ancylostoma is primarily found in southern regions. Therefore, a mammal would meet at least one evaluation metric if it fell into any of these geographical categories.

In an aspect, the evaluation metric is the level of care experienced by the mammal. For example, there is a higher likelihood of intestinal parasite infections in stray or feral dogs and cats than in cared-for cats and dogs. There is also a lower likelihood of intestinal parasite infections in dogs and cats that are given periodic wellness tests and/or anthelmintic treatment for such infections. Greyhounds kept in a racing environment have an increased likelihood of infection with hookworm. Therefore, a mammal that is not well cared for would meet at least one evaluation metric.

In an aspect, the evaluation metric is one or more symptoms that are consistent with intestinal parasite infection in the mammal. Such symptoms include, e.g., loose stool, diarrhea, vomiting, weight loss, blood in stool, or a combination thereof. Therefore, a mammal that has one or more symptoms of an intestinal parasite would meet at least one evaluation metric.

Therefore, an evaluation metric can be considered as a risk factor for intestinal parasite infection. Any known risk factor for intestinal parasites can be used as an evaluation metric.

In an aspect, the mammal can be a dog or cat, and the age of the mammal can be less than about 5 years, less than about 4 years, less than about 3 years, less than about 2 years, or less than about 1 year. The age of the dog can be about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months or more.

A mammal can be considered to have an intestinal parasite infection if the signal output from the first test is greater than the first threshold value and/or if the second test yields a positive result. The methods can be used to determine that the mammal does not have an intestinal parasite infection if the signal output from the first test is less than the first threshold value.

In some aspects, the methods can be used to determine that the mammal has an intestinal parasite infection if the signal output from the first test is greater than the first threshold value and the second test yields a positive result.

In some aspects, the methods can be used to determine that the mammal has an intestinal parasite infection if the signal output from the first test is greater than the first threshold and the second test yields a negative result.

In some aspects, the methods can be used to determine that the mammal has an intestinal parasite infection if the signal output from the first test is less than the first threshold value, but the mammal meets at least one evaluation metric and the second test yields a positive result.

In some aspects, the methods can be used to determine that the mammal does not have an intestinal parasite infection if the signal output from the first test is less than the first threshold value but the mammal meets at least one evaluation metric and the second test yields a negative test result.

In some aspects, the methods can be used to determine that the mammal does not have an intestinal parasite infection if the signal output from the first test is greater than the first threshold value, but less than the second threshold value and the second test yields a negative result.

In some aspects, the methods can be used to determine that the mammal has an intestinal parasite infection if the signal output from the first test is greater than both the first threshold value and the second threshold value and the second test yields a positive result.

In some aspects, the methods can be used to determine that the mammal has an intestinal parasite infection if the signal output from the first test is greater than both the first threshold value and the second threshold value and the second test yields a negative result.

In some aspects, the methods can be used to determine that the mammal has an intestinal parasite infection if the signal output from the first test is greater than the first threshold value but less than the second threshold value and the second test yields a positive result.

In some aspects, each of the intestinal parasites evaluated in the first test can have their own first and second threshold values. In some aspects, the first and second threshold values for each intestinal parasite can be adjusted based on the one or more evaluation metrics. In some aspects, the second test can be conducted if the signal output is greater than the first threshold value for at least one intestinal parasite. As a non-limiting example, the first threshold value for Giardia can be an MFI of about 3,700, 3,800, 4,000, 4,200, 4,400, 4,600, 4621, 4,800, 5,000, 5,200, 5,400, or 5,500 RFU. As a non-limiting example, the first threshold value for hookworm can be an MFI of about 3,600, 3,800, 4,000, 4,200, 4,500, 4,800, 5,000, 5,200, or 5,400 RFU. As a non-limiting example, the first threshold value for roundworm can be an MFI of about 600, 700, 750, 800, or 900 RFU. As a non-limiting example, the first threshold value for whipworm can be an MFI of about 600, 700, 750, 800, or 900 RFU. In some aspects, the first threshold value for any intestinal parasite can be an MFI of about 250, 300, 400, or 500 RFU. In an aspect, a low threshold value is one that is in the lower 20% of the value ranges listed above. In an aspect, a high threshold value is one this is in the upper 20% of the value ranges listed above. For example, a low threshold value for hookworm would be about 3,600 to about 3,960 RFU and a high threshold value would be about 5,040 to about 5,400 RFU.

As a non-limiting example, the second threshold value for Giardia can be an MFI of about 3,700, 3,800, 4,000, 4,200, 4,400, 4,600, 4621, 4,800, 5,000, 5,200, 5,400, or 5,500 RFU. As a non-limiting example, the second threshold value for hookworm can be an MFI of about 5,000, 5,200, 5,400, 5,600, 5,800, 6,000, 6,200, 6,303, 6,400, 6,600, 6,800, 7,000, 7,200, 7,400, or 7,500 RFU. As a non-limiting example, the second threshold value for roundworm can be an MFI of about 1,300, 1,500 1,600, 1,719, 1,800, 2,000, or 2,100 RFU. As a non-limiting example, the second threshold value for whipworm can be an MFI of about 4,900, 5,000, 5,200, 5,400, 5,600, 5,800, 6,000, 6,168, 6,200, 6,400, 6,600, 6,800, 7,000, 7,200, or 7,400 RFU. As a non-limiting example, the second threshold value for tapeworm can be an MFI of about 400, 450, 500, 550 or 600 RFU.

In an aspect, a high threshold value is one this is in the upper 20% of the value ranges listed above. For example, a low threshold value for hookworm would be about 5,000 to about 5,500 RFU and a high threshold value would be about 7,000 to about 7,500 RFU.

In another aspect, a first threshold value for hookworm can be set at about 91% sensitivity and about 98% specificity and a second threshold value for hookworm can be set at about 80% sensitivity and about 99% specificity. A first threshold value for roundworm can be set at about 94% sensitivity and about 98.4% specificity and a second threshold value for roundworm can be set at about 90% sensitivity and about 99% specificity. A first threshold value for whipworm can be set at about 90% sensitivity and about 99% specificity and a second threshold value for whipworm can be set at about 66% sensitivity and about 100% specificity. High and low threshold values can be calculated the same for any threshold range or value provided herein.

In an aspect, a first threshold value for any parasite can be set at about 55, 60, 65, 70, 80, 85, 90, 95, or 99.9% sensitivity and about 80, 85, 90, 95, or 100% specificity and a second threshold value for any parasite can be set at about 60, 65, 70, 75, 80, 85, 90, 95, or 99.9% sensitivity and about 85, 90, 95, or 100% specificity. In an aspect, a high threshold value is one this is in the upper 20% of the value ranges listed above. For example, a low first threshold value for sensitivity would be about 55 to 66.98% and a high first threshold value for sensitivity would be about 89.92 to about 99.9%. In a further example, For example, a low second threshold value for sensitivity would be about 80-84% and a high second threshold value for sensitivity would be about 96 to about 100%. High and low threshold values can be calculated the same for any threshold range or value provided herein.

TABLE 1
Diagnostic workflows

| | | | | | Intestinal |
|---|---|---|---|---|---|
| | | | | | Parasite |
| | First Test, | First Test, | | | Infection |
| Diagnostic | First | Second | Evaluation | Second | Status |
| Workflow | Threshold | Threshold | Metric | Test | (overall |
| No. | (result) | (result) | Present? | (result) | result) |
| 1 | Negative | (Not | No | Not | Negative |
| | | applied) | | performed | |
| 2 | Positive | (Not | No | Positive | Positive |
| | | applied) | | | |
| 3 | Positive | (Not | No | Negative | Positive |
| | | applied) | | | |
| 4 | Negative | (Not | Yes | Negative | Negative |
| | | applied) | | | |
| 5 | Negative | (Not | Yes | Positive | Positive |
| | | applied) | | | |
| 6 | Positive | Negative | No | Negative | Negative |
| 7 | Positive | Positive | No | Positive | Positive |
| 8 | Positive | Positive | No | Negative | Positive |
| 9 | Positive | Negative | No | Positive | Positive |

The intestinal parasite infection status as determined by various exemplary diagnostic workflows is shown in Table 1.

### Immunoassay

In an aspect, a first test can be an immunoassay. The immunoassay can utilize antibodies and/or specific binding fragments thereof to capture and detect one or more antigens of one or more intestinal parasites. In an aspect, the antibodies or specific binding fragments thereof can specifically bind to an intestinal parasite antigen and can be used to specifically identify the type of intestinal parasite. An antibody or specific binding fragment thereof can be a monoclonal antibody, polyclonal antibody, a Fab, a Fab′, a F(ab′)2, a variable fragment (Fv), a disulfide-stabilized Fv (dsFv), a single-chain Fv (scFv), a nanobody, a diabody, a triabody, an immunoglobulin single variable domain (ISV), or an AFFIBODY®.

Immunoassays for detection of hookworm are described in U.S. Pat. Nos. 9,239,326 and 8,895,294. Immunoassays for detection of roundworm are described in U.S. Pat. Nos. 9,212,220; 9,103,823, 8,105,795; and 8,895,294. Immunoassays for detection of whipworm are described in U.S. Pat. No. 8,367,808 and/or U.S. Pat. No. 8,895,294. Immunoassays for detection of tapeworm are described in U.S. Pat. No. 11,001,626. Immunoassays for detection of Giardia are disclosed in H. Stibbs, J. Clin. Microbiol., (11):2582-2588, November 1989 and H. Stibbs et al., J. Clin. Microbiol., (10):2340-6, October 1990 (the contents of each of which are herein incorporated by reference in its entirety).

The antibody-antigen complex formed in the presence of the antigen of the intestinal parasite, if any, can be detected by measuring the signal output from the first test. Detecting the presence or absence of the antigen-antibody complex can optionally further include a step of providing at least one secondary antibody that binds to the complex.

The antibodies that bind to the antigen or secondary antibodies can be attached to a substrate and/or a solid support. A substrate or solid support can be, for example, an epoxy-based resin or a solid support coated with the epoxy-based resin. Illustrative solid support materials upon which the epoxy-based resin can be coated include, but are not limited to, particles, beads and surfaces comprising glass, polymers, latex, elemental metals, metal composites, alloys, silicon, carbon, and hybrids thereof. Suitable epoxy-based resins include, but are not limited to, EPON SU-8, EPON 1001F, 1002F, 1004F, 1007F, 1009F, 2002, and 2005 (commercially available from Hexion Specialty Chemicals of Fayetteville, N.C.). A substrate or solid support can include a surface that is an epoxy-based resin. The substrate or solid support that has a surface comprising an epoxy-based resin can be, but is not limited to, a film, alone or adhered to another solid surface; a microbead; a microparticle; a micro pellet; a microwafer; a paramagnetic bead; a microparticle containing an identifying feature, such as a bar code; a paramagnetic microparticle; a paramagnetic microparticle containing a bar code; and a bead containing a nickel bar code. In an aspect, antibodies can be directly bonded to the epoxy-based resin. The substrate or solid support can, for example, have a surface comprising an epoxy-based resin that is a barcoded magnetic bead, such as a barcoded magnetic bead coated with SU-8 epoxy-based negative photoresist (commercially available from Applied BioCode of Santa Fe Springs, California).

An immunoassay can be based on a barcoded magnetic bead technology (BMB). BMB-based immunoassays are described in, for example, U.S. Patent Publication 2022/0283159 and/or in Esty et al. (J. of Vet. Diagnostic Investigation, 2023, Vol. 35(1) 57-61), which can be used in the present methods. The BMBs can comprise about 40×65×5 μm wafer-like particles with functionalized surfaces for attachment of antibodies. Each bead can include a digital barcode bonded to its surface using a semiconductor lithography process that can allow its identification by brightfield microscopy. The magnetic property of bead can permit routine processing in a 96-well microtiter plate, and can be adapted for automated, high throughput screening. Antibody-antigen complex formed in the presence of the antigen of the intestinal parasite can be detected using a detection agent such as a fluorescent label, and the signal output can be quantified across each set of unique BMBs. Unique BMBs for each antigen tested, thus representing a different assay, can be added to a single well of the microtiter plate.

Antibodies of an immunoassay can bind to an antigen that is a protein of an intestinal parasite, such as a round worm, a whip worm, a hookworm, a tape worm, a heart worm, or the parasite Giardia. One or more antibodies utilized in an immunoassay can be specific for coproantigens of an intestinal parasite. A coproantigen can be any intestinal parasite product that is present in the feces of a mammal having an intestinal parasite infection which can be specifically bound by one or more of the antibodies. Coproantigen assays have been developed for the diagnosis of a range of human and animal intestinal infections. In general antibodies raised against whole parasite extracts are coated on microtiter plates, and subsequently fecal antigen is captured and detected with the same or second parasite-specific antibody in a capture assay. The use coproantigen assays can increase both the sensitivity and the specificity of such assays as compared to floatation type assays.

Coproantigen detection methods are advantageous in that they do not rely on an amplification process such as PCR and are therefore less likely to have false positive results due to contamination of samples or reactions. Additionally, Coproantigen assays can detect early stages of infection prior to the parasite producing eggs. Therefore, coproantigens can be detected before eggs.

Coproantigen assays have been developed for, e.g., hookworms (Bungiro & Cappello, Am J Trop Med Hyg. 2005 November; 73(5):915-20), A. lumbricoides (Lagatie et al., PLoS Negl Trop Dis. 2020 Oct. 15; 14(10):e0008807. doi: 10.1371/journal.pntd.0008807) and S. stercoralis (El-Badry, J Egypt Soc Parasitol. 2009 December; 39(3):757-68; Sykes & McCarthy, PLoS Negl Trop Dis. 2011 Feb. 8; 5(2):e955. doi: 10.1371/journal.pntd.0000955). Elsemore et al. (J Vet Diagn Invest. 2017 September; 29(5):645-653. doi: 10.1177/1040638717706098) developed coproantigen tests for detecting T. canis, T. cati, Ancylostoma tubaeforme, and A. caninum using polyclonal and monoclonal antibodies for A. caninum Asp-5 protein and a T. canis protease inhibitor homologue. Elsemore et al. (J Vet Diagn Invest. May; 26(3):404-41 (2014)) designed an ELISA for coproantigen detection of infection using monoclonal antibodies for recombinant whipworm (Trichuris vulpis) porin protein. Three nematode species can be detected using the coproantigen ELISA Faecal Dx™ antigen testing from IDEXX Laboratories, Inc.

An immunoassay can include antibodies that specifically bind a coproantigen from roundworm, antibodies that specifically bind a coproantigen from whipworm, antibodies that specifically bind a coproantigen from hookworm, antibodies that specifically bind a coproantigen from tapeworm, and/or antibodies that specifically bind a coproantigen from Giardia.

Antibodies for use in the immunoassays of the disclosure can bind to a coproantigen from hookworm. For example, the antibodies can bind to sequences numbered 3 and 4 of U.S. Pat. No. 9,239,326 or a portion thereof, and/or sequences numbered 33-34 of U.S. Pat. No. 8,895,294. In some aspects, the antibodies for use in the immunoassays of the disclosure can bind to a coproantigen from roundworm. For example, the antibodies can bind to sequences numbered 3-7 of U.S. Pat. No. 9,212,220; and/or sequences numbered 3-7 and 11 of U.S. Pat. No. 9,103,823; and/or sequences numbered 3-7 of U.S. Pat. No. 8,105,795; and/or sequences numbered 12-16; 19-23; 26-30; 38 of U.S. Pat. No. 8,895,294. Antibodies for use in the immunoassays can bind to a coproantigen from a whipworm. For example, the antibodies can bind to sequences numbered 3-9 of U.S. Pat. No. 8,367,808 and/or sequences numbered 3-9 of U.S. Pat. No. 8,895,294. Antibodies for use in the immunoassays can bind to a coproantigen described in U.S. Pat. No. 11,001,626. Examples of antibodies that specifically bind coproantigens from Giardia are disclosed in H. Stibbs, Monoclonal antibody-based enzyme immunoassay for Giardia lamblia antigen in human stool, J. Clin. Microbiol., (11):2582-2588, November 1989 and H. Stibbs et al., Identification of Giardia lamblia-specific antigens in infected human and gerbil feces by western immunoblotting, J. Clin. Microbiol., (10):2340-6, October 1990.

### Nucleic Acid Assays

In some aspects, the first test can be a nucleic acid assay. As used herein, the term “nucleic acid or nucleic acid molecule” is synonymous with, and therefore used interchangeably with, “gene”, “DNA”, “cDNA”, “EST”, “polynucleotide”, “oligonucleotide”, “polynucleic acid”, “RNA” and “mRNA”. A nucleic acid molecule can be in double-stranded form, or it can be in single-stranded form. Further, a nucleic acid molecule can be either naturally isolated, such as from an intestinal parasite, for example, or it is artificially synthesized, either in a recombinant host organism or by any other artificial means known to the skilled artisan, such as by employing a PCR-based technique, by using a DNA synthesizing machine, or by any another molecular-based technique, for example. As used herein the term nucleic acid molecule can be used to refer to both a nucleic acid molecule of an intestinal parasite and a nucleic acid probe or primer as described herein.

A nucleic acid assay can be used to determine the presence or absence of a nucleic acid molecule of one or more intestinal parasites (also referred to herein as the target nucleic acid molecule). A target nucleic acid molecule of an intestinal parasite can be unique to an intestinal parasite type. Therefore, a specific intestinal parasite type can be detected and identified. The nucleic acid assay can be, for example, a polymerase chain reaction (PCR)-based assay.

Polymerase chain reaction amplification (PCR) is an amplification method in which thermal cycling, consisting of cycles of repeated heating and cooling of the reaction for nucleic acid molecule melting and enzymatic replication of the nucleic acid molecules. Typically, PCR consists of a series of 20-40 cycles. For example, the cycle can include a denaturation step, an annealing step (allowing annealing of the primers to the single-stranded DNA or RNA template) and an extension/elongation step. Each step can occur at a particular temperature, for a particular length of time, and under particular reaction conditions. For example, the temperature of the denaturation step can be about 80, 85, 90, 94, 95, 96, 97, 98, 99, or 100° C., the temperature of the annealing step can be about 40, 50, 55, 60, 62, 65, or 70° C., and the temperature of the extension/elongation step can be about 40, 50, 55, 60, 65, 70, 72, or 75° C. Exemplary PCR-based techniques are described in, e.g., PCR Protocols (Methods in Molecular Biology), 2nd ed., Bartlett and Stirling, eds., Humana Press (2003); and Sambrook and Russell, Molecular Cloning: A Laboratory Manual, Cold Spring Harbor Laboratory Press (2001); each one of which is incorporated herein by reference in its entirety.

A PCR assay can be a real-time polymerase chain reaction, which can be quantitative, and can also be combined with a reverse transcriptase reaction when performing reverse transcription-PCR. Other types of PCR assays that can be used herein include, but are not limited to, hot start PCR, reverse transcription polymerase chain reaction, multiplex-polymerase chain reaction, miniprimer polymerase chain reactions, solid phase polymerase chain reaction, digital PCR reaction, droplet PCR reaction, touchdown polymerase chain reaction. Combinations of one or more of these techniques can also be used.

A PCR-based assay can be a real-time PCR assay. Real-time PCR permits the specific amplification and detection of target nucleic acid molecules. The technique relies on a primer/probe detection mix that specifically targets a target nucleic acid molecule's sequence. In the presence of a target nucleic acid molecule (e.g., a nucleic acid molecule of an intestinal parasite), each round of PCR amplification results in an increasing signal, for example, a fluorescent signal that can optionally be detected real-time.

A PCR-based assay for detection of roundworms can be as described by Phuphisut O. et al. (Southeast Asian J Trop Med Public Health. 2014; 45: 267-275); Llewellyn S et al. (PloS Negl Trop Dis. 2016; 10: e0004380); Pilotte et al. (PloS Negl Trop Dis. 2016; 10: e0004578) and/or Wang et al. (Parasitol Int. 2018 October; 67(5):622-626). PCR-based assay for detection of Giardia can be as described by Wang et al. (Clin Microbiol. 2004; 42: 3262-3271). PCR-based assay for detecting hookworm can be as described by Massetti et al. (PLoS Negl Trop Dis. 2020 Jun. 15; 14(6): e0008392). PCR-based assay for detecting tapeworms can be as described by Labuschagne M et al. Parasite. 2018; 25:30, Kolapo T U et al. (J Parasitol. 2021; 292:109400) and/or Beugnet F et al. (Vet Parasitol. 2014; 205(1-2):300-306). PCR-based assays for detecting whipworm can be as described by Areekul et al. Asian Biomedicine Vol. 4 No. 1 Feb. 2010; 49-60 (the contents of each of which are herein incorporated by reference in their entirety).

A PCR-based assay can be a multiplex assay in which target nucleic acid molecules of more than one intestinal parasite can be amplified in the same assay. A multiplex PCR-based assay can detect hookworm, Giardia, roundworm, whipworm, tapeworm and others. As a non-limiting example, a multiplex PCR-based assay can be a commercially available assay such as, but not limited to, KeyScreen™ GI Parasite PCR assay available from Antech Diagnostics (Fountain Valley, California), FilmArray® GI available from BioFire Diagnostics (Salt Lake City, Utah), xTAG® GPP available from Luminex Corporation (Austin, Texas), FTD® Gastroenteritis available from Fast-track Diagnostics (Luxembourg), RIDA® GENE Parasitic Stool Panel available from R-Biopharm (Germany), EasyScreen™ available from Genetic Signatures (Australia), GastroFinder™ available from PathoFinder (Netherlands), BD MAX™ Enteric Parasite Panel available from Becton, Dickinson and Company (USA), and/or OpenArray™ nanolitre real-time PCR platform available from Life Technologies Corporation (Carlsbad, California).

A nucleic acid assay can utilize a primer and/or a probe. A primer, as used herein, is a polynucleotide that is capable of hybridizing with a target nucleic acid molecule and serves as an initiation site for nucleotide (RNA or DNA) polymerization under suitable conditions (e.g., in the presence of four different nucleoside triphosphates and a thermostable enzyme for polymerization, such as DNA polymerase or reverse transcriptase) in a buffer and at a suitable temperature. A primer need not have the exact sequence of a target nucleic acid molecule but must be sufficiently complementary to hybridize with a polynucleotide. A primer binding site is the region of a target nucleic acid molecule to which a primer hybridizes. A probe, as used herein, is a nucleic acid molecule capable of binding to a target nucleic acid molecule of complementary sequence through one or more types of chemical bonds, e.g., through hydrogen bond formation, such that a duplex structure is formed. A probe binds or hybridizes to a probe binding site. The probe can be labeled with a detection agent to permit detection of the probe, particularly after the probe has hybridized to its complementary target nucleic acid molecule. Alternatively, a probe can be unlabeled, but can be detectable by specific binding with a ligand that is labeled, either directly or indirectly. Moreover, a labeled probe can be detected after being hydrolyzed by an enzyme having an exonuclease activity.

A primer or probe sequence can be 100% complementary to the target nucleic acid molecule to which it hybridizes or can be less than 100% complementary. In certain aspects, a primer or probe sequence has at least 65% identity to the complement of the target nucleic acid molecule sequence of an intestinal parasite, over a sequence of at least about 5, 6, 7, 8, 9, or 10 nucleotides, more typically over a sequence in the range of 10-30 nucleotides, and often over a sequence of at least 14-25 nucleotides, and more often has at least 75% homology, at least 85% homology, at least 90% homology, or at least 95%, 96%, 97%, 98%, or 99% homology.

The target nucleic acid molecule of one more intestinal parasites can form a complex with the nucleic acid primer or probe. The presence of the nucleic acid molecule of the intestinal parasite, if any, can be detected by measuring the signal output of the assay. The nucleic acid assay can utilize one or more nucleic acid probes attached to a substrate or solid support. A nucleic acid assay can be used to distinguish between one or more intestinal parasites.

The primer or probe utilized in the nucleic acid assay can be attached to a substrate and/or a solid support. A substrate or solid support can be, for example an epoxy-based resin. A substrate can be a solid support coated with an epoxy-based resin. Illustrative solid support materials upon which the epoxy-based resin can be coated include, but are not limited to, particles, beads and surfaces comprising glass, polymers, latex, elemental metals, metal composites, alloys, silicon, carbon, and hybrids thereof. Suitable epoxy-based resins include, but are not limited to, EPON SU-8, EPON 1001F, 1002F, 1004F, 1007F, 1009F, 2002, and 2005 (commercially available from Hexion Specialty Chemicals of Fayetteville, N.C.). A substrate or solid support can include a surface that is an epoxy-based resin. The substrate or solid support that has a surface comprising an epoxy-based resin can be, but is not limited to, a film, alone or adhered to another solid surface; a microbead; a microparticle; a micro pellet; a microwafer; a paramagnetic bead; a microparticle containing an identifying feature, such as a bar code; a paramagnetic microparticle; a paramagnetic microparticle containing a bar code; and a bead containing a nickel bar code. A nucleic acid probe or primer can be directly bonded to the epoxy-based resin. A substrate or solid support can have a surface comprising an epoxy-based resin that is a barcoded magnetic bead, such as a barcoded magnetic bead coated with SU-8 epoxy-based negative photoresist (commercially available from Applied BioCode of Santa Fe Springs, California).

In an aspect, a nucleic acid assay can be based on a barcoded magnetic bead technology (BMB). An example of a BMB-based assay is described in U.S. Patent Publication 2022/0283159 and/or in Esty et al. (J. of Vet. Diagnostic Investigation, 2023, Vol. 35(1) 57-61) and can be used in the present disclosure. The BMBs can comprise about 40×65×5 μm wafer-like particles with functionalized surfaces for attachment of nucleic acid probes, primers and/or target nucleic acid molecule. Each bead can include a digital barcode bonded to its surface using a semiconductor lithography process that can allow its identification by brightfield microscopy. The magnetic property of bead can permit routine processing in a 96-well microtiter plate, and can be adapted for automated, high throughput screening. Target nucleic acid molecule-probe/primer complex formed in the presence of the target nucleic acid molecule can be detected using a detection agent such as a fluorescent label, and the signal output can be quantified across each set of unique BMBs. A fluorescent label can bind to the complex of target nucleic acid molecule and nucleic acid probe/primer. Unique BMBs for each target nucleic acid molecule tested, thus representing a different assay, can be used in a multiplex assay format.

A concentrated fecal sample, concentrated parasites, concentrated eggs, concentrated cysts or combinations thereof can be used as the sample input for the first or second test. A concentrated fecal sample, concentrated parasites, concentrated eggs, concentrated cysts, fragments or combinations thereof can be used as the sample input, i.e., as the source of template nucleic acid molecule, in a nucleic acid assay, e.g., a PCR assay.

A nucleic acid assay can be employed as the first and/or the second test. Intestinal parasites can be resistant to anthelmintics. For example, hookworms can be resistant to benzimidazole. A nucleic acid assay can be used to determine the presence or absence of anthelmintic resistance markers. Anthelmintic resistance markers can be mutations, variants, and/or gene expression levels. Anthelmintic resistance markers are known to those skilled in the art. Anthelmintic resistance markers in hookworm and tapeworm are described in Evason, M. D., et al. (2023). Emergence of canine hookworm treatment resistance: Novel detection of Ancylostoma caninum anthelmintic resistance markers by fecal PCR in 11 dogs from Canada. American Journal of Veterinary Research, 84(9), ajvr.23.05.0116, and Jimenez Castro P D et al., Multiple drug resistance in the canine hookworm Ancylostoma caninum: an emerging threat?Parasit Vectors. 2019 Dec. 9; 12(1):576. doi: 10.1186/s13071-019-3828-6, and Jesudoss Chelladurai J, et al., Praziquantel Resistance in the Zoonotic Cestode Dipylidium caninum. Am J Trop Med Hyg. 2018 November; 99(5):1201-1205. doi: 10.4269/ajtmh.18-0533 (the contents of each of which are herein incorporated by reference in its entirety).

In some aspects, the first test can be an immunoassay that is followed by a nucleic acid assay as the second test.

### Microscopic Evaluation Assay

In an aspect, a second test can be a microscopic evaluation assay. A second test can detect one or more parasite species that are not detectable by the first test e.g., Cystoisospora. The second test can involve examination of the fecal sample under a microscope for the visual detection of an intestinal parasite, including but not limited to, nematodes, capallarids, trematodes, lungworms, protozoa, stomach worms, strongylids, and tapeworms.

A capallarid can be, for example, Eucoleus aerophilus and/or Eucoleus boehmi. A trematode can be, for example, Alaria spp., Paragonimus kellicotti, Nanophyetus salmincola, Heterobilharzia americana, and/or Platynosomum fastosum. A lungworm can be, for example, Aelurostrongylus abstrusus, Angiostrongylus vasorum, Angiostrongylus cantonensis, and/or Troglostrongylus brevior.

A protozoan can be, for example, Sarcocystis spp, Neospora caninum, Hammondia heydorni, and/or Toxoplasma gondii. A stomach worm can be, for example, Physaloptera rara, Physaloptera preputialis, Ollulanus tricuspis, and/or Spirocerca lupi. A strongylid can be, for example, Strongyloides stercoralis.

A tapeworm can be, for example, Diphyllobothrium latum, Diphyllobothrium dendriticum, Echinococcus granulosus, Echinococcus multilocularis, Moniezia spp., Anoplocephala spp., Spirometra spp. and/or Mesocestoides spp.

In some aspects, a smear of the fecal sample can be prepared for microscopic evaluation. The fecal sample can be mixed with saline and spread across the surface of a microscope slide. A coverslip can be placed over the smear and the specimen can be examined microscopically for intestinal parasites, cysts, ova or fragments thereof.

Prior to the microscopic evaluation, the fecal sample can be concentrated or separated from fecal debris using a floatation method. In this method, the fecal sample is made relatively dense so that the ova, and/or parasites, and/or cysts will float to the surface of the sample. The floatation method can be used to concentrate the intestinal parasites, cysts, eggs of intestinal parasites or fragments thereof. The floatation methods can be used to separate parasites from other objects in the feces based on their differential densities. A floatation solution can be used in the floatation methods described herein. The fecal sample can be dispersed in the floatation solution. The floatation solution can be a soluble preparation of either sugar or salt in water. As a non-limiting example, the floatation solution can be sodium nitrate or sucrose.

A passive floatation method or a centrifugation-based floatation method can be applied to a fecal sample. In passive floatation method, intestinal parasites, ova or cysts whose densities are less than the density of the flotation solution are expected to rise to the surface. The floatation method can involve centrifugation, which uses centrifugal force to separate objects from intestinal parasites, cysts, eggs or fragments thereof. Centrifugation can be performed using a swinging bucket or fixed-angle rotor centrifuge.

A cover slip can be applied to the top of the floatation solution to retrieve the concentrated intestinal parasites, cysts, eggs or fragments thereof. The coverslip can then be removed and scanned for parasites under the microscope.

The fecal sample can be mixed with a stain to improve the visual observation of the intestinal parasite e.g., trichrome, Giemsa staining, hematoxylin and eosin staining.

A fecal sample can be concentrated using devices. Such devices can optionally concentrate the parasites, eggs, cysts, or fragments thereof into a pellet at the bottom of a sample tube.

Under the microscope, the type of ova, cysts, and/or the specific intestinal parasites present in the sample can be identified, and the seriousness of the infection can be determined by counting and recording the number of each type of ova, cysts and/or the specific intestinal parasites.

The microscopic evaluation assay for Giardia can involve wet mount preparations of fecal samples. Trichrome or iron hematoxylin stained preparations of fecal samples can be analyzed for Giardia after sedimentation/concentration (Momčilović S et al. Clin Microbiol Infect. 2019 March; 25(3):290-309; the contents of which are herein incorporated by reference in its entirety). The microscopic evaluation assay for roundworms can include wet mount preparation of fecal samples and/or the Kato-Katz method (Schmitt B. H. et al. Infect Dis Clin North Am. 2012; 26: 513-554). The microscopic evaluation assay for tapeworms can include wet mount preparations of fecal samples, perianal scraping (Graham's test), and/or hematoxylin and eosin staining of gravid proglottids obtained from fecal samples (Momcilovid S et al. Clin Microbiol Infect. 2019 March; 25(3):290-309; the contents of which are herein incorporated by reference in its entirety).

### Methods of Reducing the Number of Fecal Samples Subjected to Intestinal Parasite Microscopic Evaluation Assays

Provided herein are methods for reducing an amount of fecal samples in a group of fecal samples required to be subjected to intestinal parasite microscopic evaluation assays. The methods can comprise obtaining a group of fecal samples from mammals such as cats and/or dogs. For each fecal sample in the group of fecal samples a panel of tests comprising a first test and a second test are provided. The first test can be an immunoassay or a nucleic acid assay having a first threshold value and the second test can be a microscopic evaluation assay. The first test is conducted and a signal output is determined. The second test is conducted if the signal output is greater than the first threshold value or if the mammal meets at least one evaluation metric. The mammal is determined to have an intestinal parasite infection if the signal output from the first test is greater than the first threshold value, if the second test yields a positive result, or both the signal output from the first test is greater than the first threshold value and the second test yields a positive result. The amount of amount of fecal samples in the group of fecal samples subjected to intestinal parasite microscopic evaluation assays is reduced as compared to methods that do not include the methods as described herein. For example, a group of fecal samples can include about 10, 20, 30, 50, 100, 200, 500, 1,000 or more fecal samples. The amount of samples that are subjected to the second test, e.g., the microscopic evaluation assay, can be reduced by 20, 30, 40, 50, 60, 70, 80, 90% or more as compared to methods that do not require the steps of the claimed methods. In an aspect, the methods have 75, 80, 85, 90, 91, 92, 93, 94, 95, 96, 97, 98, 99 or more specificity. In an aspect, the methods have 75, 80, 85, 90, 91, 92, 93, 94, 95, 96, 97, 98, 99 or more sensitivity. In an aspect, the methods retain high sensitivity and specificity even though the number of microscopic evaluation assays are reduced overall.

### Methods of Treatment

Provided herein are methods of treatment of an intestinal parasitic infection. The methods of treatment of the disclosure can include administering one or more therapeutic agents to a subject if a positive result is obtained for a first test and/or the second test as described above. The methods of treatment described herein can include administering one or more therapeutic agents to a mammal if indicated by any of the methods described herein. When a caregiver of a mammal determines (or is otherwise informed that) that a mammal harbors an intestinal parasite infection, the caregiver may then subject the mammal to a course of treatment that is optimally designed to rid the mammal of the identified intestinal parasite specifically, rather than of a parasitic infection generally.

Treatment for roundworm infection can include the use of therapeutic agents such as, but not limited to, albendazole, ivermectin, febantel, fenbendazole, milbemycin, milbemycin oxime, moxidectin, piperazine, pyrantel, emodepside, eprinomectin, praziquantel, selamectin, oxibendazole, mebendazole or a combination thereof.

Treatment for hookworm infection can include the use of therapeutic agents such as, but not limited to, emodepside, eprinomectin, fenbantel, fenbendazole, ivermectin, selamectin, menbendazole, moxidectin, oxibendazole, pyrantel, milbemycin, milbemycin oxime, nitroscanate, afoxolaner, pamoate, praziquantel, imidacloprid or a combination thereof.

Treatment for whipworm infection can include the use of therapeutic agents such as, but not limited to, febantel, fenbendazole, milbemycin, moxidectin, oxantel, oxibendazole, afoxolaner, lufenuron, praziquantel, imidacloprid or a combination thereof.

Treatment for tapeworm infection can include the use of therapeutic agents such as, but not limited to, fenbendazole, praziquantel, epsiprantel, nitroscanate, febantel, eprinomectin, fipronil, methoprene or a combination thereof.

Treatment for Giardia infection can include the use of therapeutic agents such as, but not limited to, fenbendazole, metronidazole, albendazole, praziquantel, pyrantel, febantel, azithromycin, nitazoxanide, tinidazole, secnidazole, chloroquine, paromomycin, or a combination thereof.

Humans who may come in contact with the infested animal or its excretions can be advised to take precautions against acquiring the intestinal parasite. In this context, it is important to determine the worm species with high specificity, as some intestinal parasites, such as roundworms and hookworms, can cause significant disease (e.g., larval migrans) in humans, whereas other intestinal parasites do not.

The methods of the disclosure can also be used to confirm that any mammal that has received treatment for a particular intestinal parasitic infection has been rid or cured of that infection. Therefore, provided is a method of monitoring treatment of an intestinal parasite in a mammal. The method can comprise obtaining a fecal sample from a mammal that has been treated for an intestinal parasite. The treatment can have occurred 6, 5, 4, 3, 2, 1 or less months or 4, 3, 2, 1, or less weeks prior to performing the method of monitoring treatment. A panel of tests is provided, comprising a first test and a second test, wherein the first test is an immunoassay or a nucleic acid assay having a first threshold value and the second test is a microscopic evaluation assay. The first test can be conducted and a signal output from the first test can be determined. The second test can be conducted and if the signal output from the first test is greater than the first threshold value or if the mammal meets at least one evaluation metric, then is it determined that the mammal still has an intestinal parasite infection if the signal output from the first test is greater than the first threshold value, if the second test yields a positive result, or both the signal output from the first test is greater than the first threshold value and the second test yields a positive result. Where it is determined that the mammal still has an intestinal parasite infection one or more treatments for intestinal parasite infection can be administered to the mammal.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### EXAMPLES

### Example 1: Performance of Parallel Testing Using Immunoassay and Microscopic Evaluation Assay with Immunoassay and Second, Reflex Microscopic Evaluation Assay

850 fecal samples were used for side by side comparison of using (i) parallel testing with immunoassay (first test) and microscopic evaluation (second test) (ii) conducting a microscopic evaluation based on the outcome of the immunoassay. In (ii), the microscopic evaluation is a reflex test that is performed when the immunoassay meets the first threshold value. In the parallel testing using the immunoassay and the microscopic evaluation are both performed in parallel. The immunoassay in the parallel testing workflow has a threshold value designed to enhance the specificity of the immunoassay. The threshold values in MFI were as follows: 4621 (Giardia), representing 1.84% of the dynamic range; 6303 (hookworm), representing 2.52% of the dynamic range; 1719 (roundworm), representing 0.69% of the dynamic range; 6168 (whipworm), representing 2.47% of the dynamic range. Based on the observed results, the sensitivity and the specificity of the immunoassay were calculated and compared to the microscopic evaluation assay as shown in Table 2.

TABLE 2
Sensitivity and specificity of immunoassay
in comparison to microscopic evaluation assay

| Immunoassay vs. | | | | |
|---|---|---|---|---|
| microscopic | | | | |
| evaluation assay | Giardia | Hook | Round | Whip |
| Sensitivity | 97% | 88% | 86% | 54% |
| Specificity | 88% | 94% | 99% | 99% |

The prevalence of the intestinal parasites based on the results of the immunoassay and microscopic evaluation assay combined are shown in Table 3.

TABLE 3
Prevalence of intestinal parasites detected by parallel workflow

| Giardia | Hookworm | Roundworm | Whipworm |
|---|---|---|---|
| 14.5% | 15.4% | 3.4% | 3.1% |

In the workflow where the microscopic evaluation assay was a reflex test, the threshold values in MFI were as follows: 4621 (Giardia), representing 1.84% of the dynamic range, 4500 (Hook), representing 1.80% of the dynamic range, 750 (Round) representing 0.3% of the dynamic range, 750 (Whip) representing 0.3% of the dynamic range. Based on the observed results, the sensitivity and the specificity of the immunoassay were calculated and compared to the microscopic evaluation as shown in Table 3. Of the 850 samples tested, 74% of the samples (632 samples) were negative for the immunoassay, thereby reducing the number of samples requiring microscopic evaluation assay to be reduced to 241 samples.

TABLE 4
Sensitivity and specificity of immunoassay
in comparison to microscopic evaluation assay.

| Immunoassay vs. | | | | |
|---|---|---|---|---|
| microscopic | | | | |
| evaluation assay | Giardia | Hook | Round | Whip |
| Sensitivity | 97% | 90% | 86% | 92% |
| Specificity | 88% | 92% | 99% | 92% |

The prevalence of the intestinal parasites based on the results of the immunoassay and microscopic evaluation assay combined are shown in Table 5.

TABLE 5
Prevalence of intestinal parasites detected by conducting microscopic
evaluation based on the outcome of the immunoassay.

| Giardia | Hookworm | Roundworm | Whipworm |
|---|---|---|---|
| 14.5% | 16.1% | 3.5% | 8.8% |

These results show that it is possible to maintain similar prevalence to existing combined method but allowing for a 74% reduction in the use of the microscopic evaluation assay.

### Example 2: Diagnostic Workflow Case Study

An adult dog is evaluated as part of annual wellness visit. The dog is otherwise healthy. No evaluation metric is present. An immunoassay is performed on a fecal sample from the adult dog using the first threshold value. Results for all antigens tested in the immunoassay are negative. Microscopic evaluation assay is not indicated for this sample.

The report prepared for this fecal sample is as follows: No antigen detected for Giardia, ascarids, hookworm, whipworm, or Dipylidium caninum. Centrifugal flotation not indicated. Therefore, no treatment would be suggested.

### Example 3: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The fecal sample is positive in the immunoassay for hookworm. The microscopic evaluation assay is performed, and hookworm eggs are detected, moderate (5-10/hpf).

The report prepared for this fecal sample is as follows: No antigen detected for Giardia, ascarids, whipworm, or Dipylidium caninum. Antigen positive for hookworm. Centrifugal flotation performed. Hookworm eggs present, moderate (5-10/hpf). Therefore, hookworm treatment would be suggested.

### Example 4: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The fecal sample is positive in the immunoassay for hookworm. The sample is reflexed to microscopic evaluation assay, but no parasites are seen.

The report prepared for this fecal sample is as follows: no antigen detected for Giardia, ascarids, whipworm, or Dipylidium caninum. Antigen positive for hookworm. Centrifugal flotation is performed. No parasites seen. Therefore, treatment for hookworm would be suggested. In this case, it may be that this is an early infection, prior to the hookworm producing eggs.

### Example 5: Diagnostic Workflow Case Study

A young dog (12 months or younger) is evaluated as part of annual wellness visit. The dog is otherwise healthy. An evaluation metric (young age) is present. An immunoassay is performed on a fecal sample from the young dog using the first threshold value. The sample is antigen negative. The microscopic evaluation assay is performed on the sample due to the age of the animal. No parasites are seen.

The report prepared for this fecal sample is as follows: No antigen detected for Giardia, ascarids, hookworm, whipworm, or Dipylidium caninum. Centrifugal flotation performed. No parasites seen. Therefore, no treatment would be suggested.

### Example 6: Diagnostic Workflow Case Study

A young dog (12 months or younger) was evaluated as part of annual wellness visit. The dog was otherwise healthy. An evaluation metric (young age) is present. An immunoassay was performed on a fecal sample from the young dog using the first threshold value. The sample is antigen negative. Centrifugal flotation is performed on the sample due to the age of the animal. Toxocara canis eggs are present, rare (1-2/hpf).

The report prepared for this fecal sample is as follows: No antigen detected for Giardia, ascarids, hookworm, whipworm, or Dipylidium caninum. Centrifugal flotation performed. T. canis eggs present, rare (1-2/hpf). Therefore, treatment for T. canis would be suggested.

### Example 7: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The sample is antigen positive for whipworm. The second threshold value is applied to the immunoassay and the sample is negative for whipworm. The microscopic evaluation assay is performed, and no parasites are seen.

The report prepared for this fecal sample is as follows: No antigen detected for Giardia, ascarids, hookworm, whipworm, or Dipylidium caninum. Centrifugal flotation performed due to borderline antigen signal. No parasites seen. Therefore, no treatment would be suggested.

### Example 8: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The sample is antigen positive for whipworm. The second threshold value is applied to the immunoassay and the sample is antigen positive for whipworm. The microscopic evaluation assay is performed, and whipworm eggs are detected.

The report prepared for this fecal sample is as follows: No antigen detected for Giardia, ascarids, hookworm, or Dipylidium caninum. Positive for whipworm antigen. Centrifugal flotation performed. Whipworm eggs present. Therefore, treatment for whipworm would be suggested.

### Example 9: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The sample is antigen positive for whipworm. The second threshold value is applied to the immunoassay and the sample is antigen positive for whipworm. The microscopic evaluation assay is performed, and no parasites are seen.

The report prepared for this fecal sample is as follows: No antigen detected for Giardia, ascarids, hookworm, or Dipylidium caninum. Positive for whipworm antigen. Centrifugal flotation performed. No parasites seen. Therefore, treatment for whipworm would be suggested.

### Example 10: Diagnostic Workflow Case Study

An adult dog is evaluated as part of annual wellness visit. The dog is otherwise healthy. No evaluation metric is present. An immunoassay is performed on a fecal sample from the adult dog using the first threshold value. The sample is antigen positive for whipworm. The second threshold value is applied to the immunoassay and the sample is antigen negative for whipworm. The microscopic evaluation assay is performed. Whipworm eggs present.

The report prepared for this fecal sample is as follows: No antigen detected for Giardia, ascarids, hookworm, whipworm or Dipylidium caninum. Centrifugal flotation performed due to borderline antigen signal. Whipworm eggs present. Therefore, treatment for whipworm would be suggested.

1. A method for detecting an intestinal parasite infection in a mammal using a fecal sample, the method comprising
a) providing a panel of tests, comprising a first test and a second test, wherein the first test is an immunoassay or a nucleic acid assay having a first threshold value and the second test is a microscopic evaluation assay;
b) conducting the first test on the fecal sample and determining a signal output from the first test;
c) conducting the second test on the fecal sample if the signal output is greater than the first threshold value or if the mammal meets at least one evaluation metric; and
d) determining that the mammal has an intestinal parasite infection if the signal output from the first test is greater than the first threshold value, if the second test yields a positive result, or both the signal output from the first test is greater than the first threshold value and the second test yields a positive result.

2. The method of claim 1, wherein the first threshold value is a low threshold value.

3. The method of claim 1, wherein the first test further comprises a second threshold value, wherein the second threshold value is a high threshold value.

4. The method of claim 3, wherein a ratio of the second threshold value to the first threshold value is at least 1.

5. The method of claim 1, wherein the at least one evaluation metric is age of the mammal, species of the mammal, breed of the mammal, incidence of an intestinal parasite in a population of the mammal, the time of year when the test is performed on the mammal, the level of care experienced by the mammal, one or more symptoms that are consistent with intestinal parasite infection, or geographical location of the mammal.

6. The method of claim 3, further comprising determining the mammal does not have an intestinal parasite infection if:
(i) the signal output from the first test is less than the first threshold value;
(ii) the signal output from the first test is less than the first threshold value and the second test yields a negative test result, wherein the mammal meets the at least one evaluation metric; or
(iii) the signal output from the first test is greater than the first threshold value, but less than the second threshold value and the second test yields a negative result.

7. The method of claim 3, further comprising determining the mammal has an intestinal parasite infection if:
(i) the signal output from the first test is greater than the first threshold value and the second test yields a positive result;
(ii) the signal output from the first test is greater than the first threshold and the second test yields a negative result;
(iii) the signal output from the first test is less than the first threshold value and the second test yields a positive result, wherein the mammal meets the at least one evaluation metric;
(iv) the signal output from the first test is greater than both the first threshold value and the second threshold value and the second test yields a positive result;
(v) the signal output from the first test is greater than both the first threshold value and the second threshold value and the second test yields a negative result; or
(vi) the signal output from the first test is greater than the first threshold value but less than the second threshold value and the second test yields a positive result.

8. (canceled)

9. (canceled)

10. (canceled)

11. (canceled)

12. (canceled)

13. (canceled)

14. (canceled)

15. The method of claim 1, wherein the intestinal parasite infection is an infection of an intestinal parasite comprising a hookworm, a Giardia, a roundworm, a whipworm, a tapeworm or a combination thereof.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The method of claim 1, further comprising administering a therapeutic agent to treat the mammal where it is determined that the mammal has an intestinal parasite.

38. (canceled)

39. A method for reducing an amount of fecal samples in a group of fecal samples required to be subjected to intestinal parasite microscopic evaluation assays, the method comprising:
a) for each fecal sample in the group of fecal samples providing a panel of tests comprising a first test and a second test, wherein the first test is an immunoassay or a nucleic acid assay having a first threshold value and the second test is a microscopic evaluation assay;
b) conducting the first test and determining a signal output;
c) conducting the second test if the signal output is greater than the first threshold value or if the mammal meets at least one evaluation metric; and
d) determining that the mammal has an intestinal parasite infection if the signal output from the first test is greater than the first threshold value, if the second test yields a positive result, or both the signal output from the first test is greater than the first threshold value and the second test yields a positive result,
wherein the amount of fecal samples in the group of fecal samples subjected to intestinal parasite microscopic evaluation assays is reduced as compared to methods that do not include steps a)-d).

40. The method of claim 39, wherein the first threshold value is a low threshold value.

41. The method of claim 40, wherein the first test further comprises a second threshold value, wherein the second threshold value is a high threshold value.

42. (canceled)

43. (canceled)

44. The method of claim 41, further comprising determining the mammal does not have an intestinal parasite infection if:
(i) the signal output from the first test is less than the first threshold value;
(ii) the signal output from the first test is less than the first threshold value and the second test yields a negative test result, wherein the mammal meets the at least one evaluation metric; or
(iii) the signal output from the first test is greater than the first threshold value, but less than the second threshold value and the second test yields a negative result.

45. The method of claim 41, further comprising determining the mammal has an intestinal parasite infection if:
(i) the signal output from the first test is greater than the first threshold value and the second test yields a positive result;
(ii) the signal output from the first test is greater than the first threshold and the second test yields a negative result;
(iii) the signal output from the first test is less than the first threshold value and the second test yields a positive result, wherein the mammal meets the at least one evaluation metric;
(iv) the signal output from the first test is greater than both the first threshold value and the second threshold value and the second test yields a positive result;
(v) if the signal output from the first test is greater than both the first threshold value and the second threshold value and the second test yields a negative result; or
(vi) the signal output from the first test is greater than the first threshold value but less than the second threshold value and the second test yields a positive result.

46.-74. (canceled)

75. A method of monitoring treatment of an intestinal parasite in a mammal that has been treated for an intestinal parasite using a fecal sample comprising:
a) providing a panel of tests, comprising a first test and a second test, wherein the first test is an immunoassay or a nucleic acid assay having a first threshold value and the second test is a microscopic evaluation assay;
b) conducting the first test on the fecal sample and determining a signal output from the first test;
c) conducting the second test on the fecal sample if the signal output from the first test is greater than the first threshold value or if the mammal meets at least one evaluation metric; and
d) determining that the mammal still has an intestinal parasite infection if the signal output from the first test is greater than the first threshold value, if the second test yields a positive result, or both the signal output from the first test is greater than the first threshold value and the second test yields a positive result.

76. The method of claim 75, wherein where if it is determined that the mammal still has an intestinal parasite infection, providing one or more treatments for intestinal parasite infection to the mammal.

77. (canceled)

78. The method of claim 75, wherein the first threshold value is a low threshold value.

79. The method of claim 75, wherein the first test further comprises a second threshold value, wherein the second threshold value is a high threshold value.

80. (canceled)

81. (canceled)

82. The method of claim 79, further comprising determining the mammal does not have an intestinal parasite infection if:
(i) the signal output from the first test is less than the first threshold value;
(ii) the signal output from the first test is less than the first threshold value and the second test yields a negative test result, wherein the mammal meets the at least one evaluation metric; or
(iii) the signal output from the first test is greater than the first threshold value, but less than the second threshold value and the second test yields a negative result.

83. The method of a claim 79, further comprising determining the mammal has an intestinal parasite infection if:
(i) the signal output from the first test is greater than the first threshold value and the second test yields a positive result;
(ii) the signal output from the first test is greater than the first threshold and the second test yields a negative result;
(iii) the signal output from the first test is less than the first threshold value and the second test yields a positive result, wherein the mammal meets the at least one evaluation metric;
(iv) the signal output from the first test is greater than both the first threshold value and the second threshold value and the second test yields a positive result;
(v) if the signal output from the first test is greater than both the first threshold value and the second threshold value and the second test yields a negative result; or
(vi) the signal output from the first test is greater than the first threshold value but less than the second threshold value and the second test yields a positive result.

84.-112. (canceled)
